(19) United States
(12) Patent Application Publication
(10) Pub. No.: US 2016/0324890 A1
(43) Pub. Date: Nov. 10, 2016

(54) Method and Device for Preventing Microbial Resistance

(71) Applicants: Robert E. Weinstein, Miami, FL (US); Allan M. Weinstein, Potomac, MD (US)

(72) Inventors: Robert E. Weinstein, Miami, FL (US); Allan M. Weinstein, Potomac, MD (US)

(21) Appl. No.: 14/854,114

(22) Filed: Sep. 15, 2015

Related U.S. Application Data
(60) Provisional application No. 62/157,612, filed on May 6, 2015.
(60) Provisional application No. 62/173,983, filed on Jun. 11, 2015.

(51) Int. Cl. A61K 33/14; G06F 19/00; A61K 31/43; A61K 31/137; A61K 31/192

(52) U.S. Cl. CPC A61K 33/14; A61K 31/137; A61K 31/192; A61K 31/43; G06F 19/345; G06F 19/328

(57) ABSTRACT

A method to treat acute sinusitis as an alternative to prescribing an antibiotic or recommending over-the-counter medications by (1) providing a non-antibiotic treatment package containing a pharmacy-dispensed decongestant and an over-the-counter therapeutic agent for acute sinusitis that includes a topical nasal moisturizing means, and further selecting pseudoephedrine that is pharmacy-dispensed wherein the non-antibiotic treatment package is a behind-the-counter treatment package, (2) advising a patient to procure the treatment package to treat acute sinusitis as an alternative to prescribing an antibiotic or recommending over-the-counter procurement of treatment, and (3) pharmacy-dispensing the treatment package to a patient.

### BACKGROUND OF THE INVENTION

1. Field of the Invention

The present invention relates generally to microbial resistance and means and methods for reducing inappropriate antibiotic utilization.

2. Description of the Prior Art

The long, widespread, and overuse of antibiotics has made them less effective for ordinary treatments and has fostered the emergence of new infectious diseases, some of which are not amenable to antibiotic treatment. Each year in the United States, at least 2 million people become infected with bacteria that are resistant to antibiotics, and at least 23,000 people die as a direct result of these infections. Reflecting the urgency of this problem, the President of the United States signed an executive order on Sep. 18, 2014 establishing a National Task Force of Combating Antibiotic-Resistant Bacteria, co-chaired by the Secretaries of Defense, Agriculture, and Health and Human Services, and asking for a National Action Plan to address growing antimicrobial resistance.

Acute viral sinusitis refers to a disease entity characterized to inflammation of the nasal and sinus membranes caused by viral infection. Symptoms typically consist of nasal congestion, thickened nasal secretions, nasal discharge, post nasal drip, tenderness in the sinus areas, malaise and low grade fever and usually resolve in seven to ten days. Bacterial sinusitis represents secondary infection where bacteria have proliferated in the nasal sinuses promoted by persistent nasal blockage and thickened mucous that interfere with the normal flow of nasal secretions. Recent guidelines for treatment of acute sinusitis do not recommend employing antibiotics in adults during the first 7 days of infection, and recommend reserving antibiotics only for when there is a strong suspicion that the cause is bacterial and not viral.

Antibiotics are presently being regularly overprescribed for acute sinusitis. Therefore, what is needed is a method and device that reduces the over-prescribing of antibiotics for acute sinusitis.

### SUMMARY OF THE INVENTION

The present invention is directed to the overuse of antibiotics for treating acute rhinosinusitis that occurs in the course of the most common human infectious disease, acute viral respiratory infection. According to a 2012 journal article, it is estimated that antibiotics are prescribed for 80% of individuals presenting to caregivers. (See Farlie, T. et al., “National trends in visit rates and antibiotic prescribing for adults with acute sinusitis,” Archives Internal Medicine, 2012, 172(19); 1513-14). In contrast, the proportion of cases of acute sinusitis due to bacteria in primary care is cited to be probably less than 2%. (See Worrall, G., “Acute Sinusitis,” Canadian Family Physician, 2011, 57(5), 565-567). Acute sinusitis represents a considerable opportunity to improve upon antibiotic overprescribing, as it affects an estimated 35 million people each year in the United States.

What has been missing from the prior art has been an explanation for the inordinate antibiotic overprescribing that occurs in the face of general acknowledgement of the viral nature of acute sinusitis. The inventor therefore initiated a survey to try to understand the practices of primary care doctors that treat acute sinusitis. The survey disclosed that all participating doctors were experiencing being pressured by patients to prescribe an antibiotic for acute sinusitis. The frequency with which the doctors were being pressured was particularly surprising. According to the survey, the doctors reported that approximately 61% of their patients urge them to prescribe an antibiotic when seeking care for acute sinusitis. Table 1 below shows the distribution of this among practices. Notably, it can be seen that over half of surveyed practitioners reported that either 75% or 100% of their patients presently seeking care for acute sinusitis urge them for antibiotics.

TABLE 1

| Percent of | % doctors |
|---|---|
| patients | reporting |
| 100% | 6% |
| 75% | 45% |
| 50% | 36% |
| 25% | 13% |

The survey was performed in May of 2015 by Medefield America Limited, LLC of Laurel New York, a division of an international healthcare market research organization, who retains all copyrights in the data.

Non-antibiotic treatments that provide symptomatic relief and ameliorate nasal blockage and stasis of nasal secretions are commonly available over-the-counter (OTC) without a prescription for the treatment of acute sinusitis. Despite being ineffective for viral illness and despite their gratuitous use placing both patient and society needlessly at risk, antibiotics are, as noted, nonetheless being prescribed 80% of the time.

Primary care doctors were queried about recommending OTC drugs. Ninety-six percent (96%), almost all doctors asked, responded to being hesitant to recommend them. A variety of reasons were cited, including expectations (1) that their patients do not regard OTC treatments as being as effective as prescription medications, (2) that patients may not buy them because of out-of-pocket expense, (3) that patients might become confused by similar OTC drugs such that the intended drug would not be procured, and (4) the pharmacy may not have the intended item. This indicates that an acceptable alternative to prescribing an antibiotic is problematic to almost all practitioners.

At present, doctors face the dilemma of having to choose between prescribing an antibiotic and advising a patient to procure an OTC drug, where eighty percent (80%) of the time the antibiotic gets prescribed. The regrettable overprescribing of antibiotics for acute sinusitis has long been well known, but frequent coercion to prescribe an antibiotic in clinical practice compounded by the hesitancy of doctors to recommend OTC drugs and the unavailability of non-OTC treatment options for acute sinusitis has not been heretofore obvious to, nor addressed by, those in the art.

To exemplify that this dilemma is not presently appreciated in the art, it can be seen that neither awareness of it, nor intentions to remedy it can be found in our largest national effort to combat microbial resistance, the National Action Plan for Combating Antibiotic-Resistant Bacteria. The Plan conducted under the auspices of the White House and updated as of March 2015 represents the current state of the art to address microbial resistance. According to page 12 of this report: “Perhaps the single most important action to slow the development and speed of antibiotic-resistant infections is to change the way antibiotics are used. Antibiotics are overprescribed . . . which makes everyone less safe. Investments in this area will be used to develop education and outreach programs to clarify and strengthen responsible, appropriate use of antibiotics in humans . . . commitment to always use antibiotics appropriately and safely . . . is known as antibiotic stewardship.” While proposing guidelines, education programs, and a Physician Quality Reporting System (PQRS), this extensive document attests to an absence of recognition of the herein-disclosed dilemma that occurs at the level of caregiver-patient interaction and sets the stage for antibiotic overprescribing.

It is difficult for caregivers to withhold antibiotics when confronted with a patient or parent who equates adequate treatment with an antibiotic and urges the caregiver for an antibiotic. Further, the caregiver is hesitant to advise the patient to seek non-antibiotic treatment over-the-counter. A patient-acceptable alternative is needed in order to shift the outcome from capitulation to a treatment alternative. Only 3% of surveyed doctors when asked, were found to disagree with the present need for a patient-acceptable alternative to an antibiotic prescription.

It is an object of the present invention to provide alternative treatments that are acceptable to present caregivers. It is another object of the present invention that the alternative treatments are anticipated by doctors to be acceptable to patients. Central to the present invention is the provision of non-antibiotic treatment packages that are necessarily dispensed by pharmacy-personnel. The present invention provides a new therapeutic option for the caregiver, where the caregiver is not limited to the choice of either prescribing an antibiotic or advising a patient to ordinarily procure an OTC drug.

It is intended for the process of conveying pharmacy-dispensed non-antibiotic treatment packages of the present invention to the patient to be akin to that of a patient acquiring a prescription drug, in contrast to a patient ordinarily procuring an OTC drug.

Further, treatment packages of the present invention are devised in accord with the treatment preferences of caregivers. Treatment preferences that caregivers have indicated more often include at least a nasal decongestant and a topical nasal moisturizing agent. It is also contemplated that the treatment packages must contain a nasal decongestant available only behind-the-counter. The term “behind-the-counter” has the meaning provided under the Combat Methamphetamine Epidemic Act of 2005, which has been included in the Patriot Bill signed by the President on Mar. 9, 2006. This is a key feature of the treatment package since a patient must go to authorized personnel in a pharmacy and request the treatment package or present a non-prescription procurement means obtained from a caregiver, where obtaining the treatment package requires purchaser identification and a purchaser's signature. It is further anticipated for such packages to preferably incorporate additional therapeutic agents for acute sinusitis as an antihistamine, an oral mucolytic agent, an analgesic, a topical corticosteroid, a topical or oral anticholinergic agent, a cough suppressant, an antiviral agent, a vitamin, an anti-inflammatory agent, and/or symptomatic vapor treatments.

In another embodiment, the present invention includes recommending the treatment package to a doctor, employing such means as are known in the art of pharmaceutical promotion, as a practice alternative to prescribing an antibiotic for acute sinusitis where the doctor does not have to direct patients to procure over-the counter medications.

It is a further embodiment of the present invention to provide a treatment package appearance commensurate with prescription packaging, and applying it to the pharmacy-dispensed non-antibiotic treatment package. The advantage of this embodiment is to appeal to the patient's perception that the treatment package is a significant treatment for acute sinusitis and comparable to a prescription treatment.

It is still another embodiment of the present invention to provide a non-prescription procurement means and conveying the non-prescription procurement means to a patient by a caregiver. The advantage of this embodiment is to also enhance the significance of the treatment package as being comparable to a drug prescription where the non-prescription procurement means is presented to the pharmacy to obtain the behind-the-counter treatment package for treating acute sinusitis.

In another embodiment, the method includes devising a restraint directive that directs a patient to employ an antibiotic for clinical contingencies of worsening as are desired to be stipulated, for example, upon encountering at least one of prolonged symptoms, worsening of symptoms after a period of improvement, high fever, severe unilateral pain in the region of the nasal sinuses, and unilateral toothache.

Further contemplated are methods to enhance the patient-acceptability of the pharmacy-dispensed antibiotic-alternative treatment packages of the present invention by providing insurance reimbursement, and by providing for the patient to have ready access to an antibiotic contingent upon encountering worsening symptoms after initial clinical encounter.

The present invention also includes providing a method to readily quantify efficacy to restrain antimicrobial use that utilizes a system of non-antibiotic and antibiotic treatment packages and tracking their relative use, and further where the system can be improved by adjusting it accordingly.

### BRIEF DESCRIPTION OF THE DRAWINGS

FIG. 1 schematically depicts an embodiment of a pharmacy-dispensed non-antibiotic sinusitis treatment package or module (NAB) according to the present invention.

FIG. 2 schematically depicts an embodiment of a sinusitis antibiotic package or module (AB) according to the present invention.

FIG. 3 exemplifies a constraint directive of the present invention.

FIG. 4 illustrates procurement means of the present invention.

FIG. 5 is a flow-sheet illustration comparing present antibiotic use to antibiotic use according to methods of the invention.

### DETAILED DESCRIPTION OF THE INVENTION

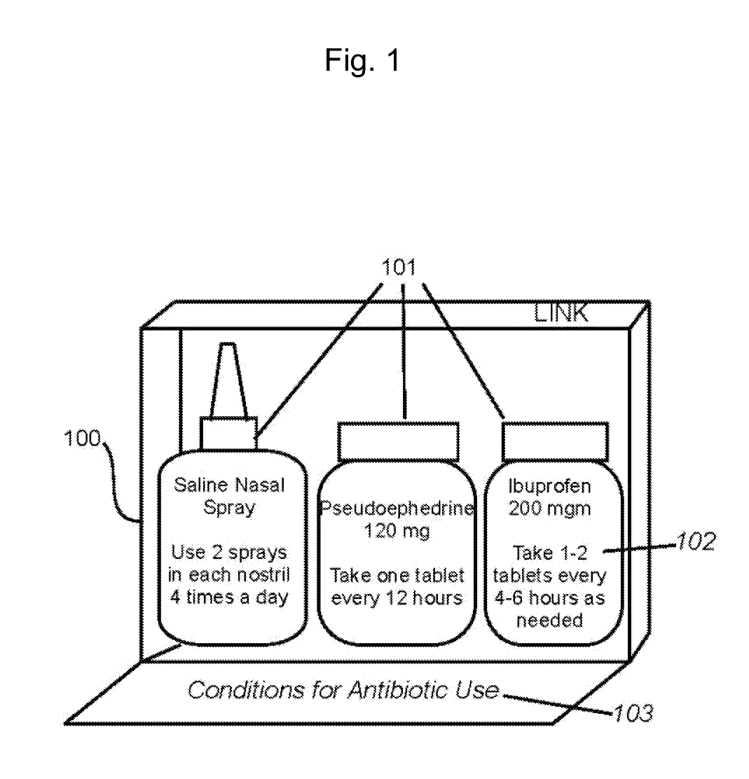

FIGS. 1-5 illustrate means and method of the present invention to aid those skilled in the art in practicing the present invention. The present invention is intended to address and improve upon overprescribing of antibiotics that is consequent to a doctor presently having to choose between prescribing and antibiotic and recommending an OTC treatment, where the antibiotic presently gets chosen. It accomplishes this by an alternative choice that is a pharmacy-dispensed sinusitis treatment package where the process of acquiring non-antibiotic sinusitis medication is akin to a prescription and unlike ordinarily procuring an OTC drug, and where doctor's expectations are for patient acceptance of this alternative.

FIG. 1 schematically depicts an embodiment 100 of a pharmacy-dispensed non-antibiotic treatment package or module according to the present invention. The package includes a nasal decongestant that is only obtainable behind-the-counter. The pharmacy-dispensed non-antibiotic treatment packages of the present invention are accordingly intended to incorporate, for example, a multiplicity of treatments that include topical nasal moisturizing means, and oral dosages having the nasal decongestant pseudoephedrine, where a patient is not required to procure an element from an over-the counter shelf on his or her own. The illustrated medications 101 include a nasal saline spray, the oral nasal decongestant pseudoephedrine, and also a third agent that is in this case the oral analgesic ibuprofen for symptomatic relief of sinus pain, headache, and associated discomfort.

The nasal decongestant pseudoephedrine is a preferred agent of the present invention based upon FDA regulations regarding its dispensing at pharmacies, where this agent is no longer dispensed OTC, but rather “behind-the-counter” by pharmacy personnel, consequent to the potential for it to be used in the illegal manufacture of methamphetamine. Congress passed the Combat Methamphetamine Epidemic Act of 2005 (“CMEA”) concerning its sale that now requires training of employees with regard to dispensing, signature of the purchaser, and storage behind the pharmacy counter to restrict public access.

It is herein recognized that this enforced conveyance can now be advantageously utilized to overcome doctors reservations about recommending ordinary OTC products to patients presenting with acute viral sinusitis. Dispensing by pharmacy personnel, even requiring the patient's signature, is akin to the process that a patient follows to acquire a prescription drug, and where the patient might view the process as prescription-like and more validating of the treatment than ordinarily procuring an OTC drug. The present invention is intended for the practitioner to have equal control of the process of the patient procuring medication as with a prescription, and pharmacy dispensing provides certainty of the patient getting the practitioner-intended treatment in contrast to purchasing OTC medication, where the patient may be confused by the array of over-the-counter medications and where there is potential for error in acquiring medicine.

The following exemplifies the potential for patient confusion in ordinarily procuring over-the-counter treatment. If a caregiver were to instruct a patient to acquire “Sudafed” at the pharmacy on his or her own, the patient might well encounter a confusing assortment of branded products, some incorporating pseudoephedrine as might be expected, but others incorporating the decongestant phenylephrine, additional ingredients, and a variety of dosing schedules: Sudafed 12 Hour, containing 120 mgm pseudoephedrine HCl, and directed to take one tablet every 12 hours, not to exceed 2 tablets in 24 hours; Sudafed 24 Hour, containing 240 mg of pseudoephedrine HCl and directed to swallow one whole tablet every 24 hours; Sudafed Congestion, containing pseudoephedrine HCl 30 mg, and directed to take 2 tablets every 4-6 hours, not to exceed 8 in 24 hours; Sudafed PE Congestion, containing phenylephrine HCl 10 mg, and directed to take one tablet every 4 hours, not to exceed 6 in 24 hours; Sudafed PE Pressure+Pain, containing acetaminophen 325 mg and phenylephrine HCl 5 mg, and directed to take 2 caplets every 4 hours, not to exceed more than 10 caplets in 24 hours; Sudafed 12 Hour Pressure+Pain, containing naproxen sodium 220 mg and pseudoephedrine HCl 120 mg, and directed to take 1 every 12 hours; Sudafed PE Pressure+Pain+Cough, containing acetaminophen 325 mg, dextromethorphan HBr 10 mg, phenylephrine 5 mg, and directed to take 2 caplets every 4 hours, not to exceed 10 in 24 hours; Sudafed PE Pressure+Pain+Cold, containing acetaminophen 325 mg, dextromethorphan HBr 10 mgm guaifenesin 100 mg, phenylephrine 5 mg, and directed to take 2 caplets every 4 hours, not to exceed 10 in 24 hours; and Sudafed PE Pressure+Pain+Mucous, containing acetaminophen 325 mg, guaifenesin 200 mg, phenylephrine HCl 5 mg, and directed to take 2 caplets every 4 hours, not to exceed 10 caplets in 24 hours. It is intended for non-antibiotic treatment packages of the present invention to overcome patient inconvenience and uncertainty by providing a physician-preferred acute viral sinusitis treatment regimen that is conveyed by authorized pharmacy personnel, and to not require patients, at a time of feeling sufficiently ill as to seek medical care, to search for unfamiliar or confusing treatments on their own.

The next most favored agents after decongestant and topical nasal moisturizing means, according to caregivers, include a mucolytic agent, an antihistamine, an analgesic, and an intranasal corticosteroid, followed by a cough suppressant and zinc lozenges. Various additional agents are accordingly contemplated for optional inclusion in non-antibiotic packages of the present invention, including decongestants other than pseudoephedrine (topical or oral), mucolytic agents (such as guaifenesin), antihistamines, analgesics (such as aspirin and NSAIDs), topical intranasal corticosteroids, cough suppressants (such as dextromethorphan), zinc (tablets, lozenges and sprays), anticholinergic agents (such as topical ipratropium, and oral methscopolamine), vitamins (such as C), anti-inflammatory agents (such as carnosine), and symptomatic vapor treatment (such as containing menthol) as might be found to suit the further needs of particular caregivers for an antibiotic-prescribing alternative.

The term “pseudoephedrine” is herein intended to refer to this agent as ordinarily pharmaceutically utilized, for example as a hydrochloride or sulfate salt. Examples of pseudoephedrine dosages commensurate with the present invention include Afrinol brand pseudoephedrine sulfate 120 mg extended release tablets manufactured by Schering-Plough and pseudoephedrine hydrochloride 120 mg extended release tablets manufactured by various generic manufacturers that are recommended for twice a day dosing, as well as pseudoephedrine presently formulated in combination with other agents such as analgesic, antihistamine, and mucolytic agents.

It is further contemplated that topical nasal moisturizing means of the present invention may be in the form of sprays, drops, and nasal washes, and comprise water, hypotonic saline, saline, hypertonic saline, and/or buffering agents, and be delivered into the nasal passages by various methods, as are known in the art. Examples of topical nasal moisturizing means commensurate with the present invention are Ocean Saline Nasal Spray and Ocean Complete Sinus Rinse, both distributed by Valeant Pharmaceuticals North America LLC, CVS Saline Nasal Spray, distributed by CVS Health, and Simply Saline Nasal Spray manufactured by Arm & Hammer, a division of Church and Dwight Corporation.

A further intention of pharmacy-dispensed sinusitis treatment packages of the present invention is to substantiate non-antibiotic treatments as caregiver-advocated treatment, and not trivial or haphazard treatment, as might appear to a patient when ordinarily procuring an OTC drug at the pharmacy. The term “pharmacy-dispensed” is herein intended to indicate conveyance to a patient by a pharmacist or by authorized pharmacy personnel, distinct from where a lay individual ordinarily procures treatments over-the-counter (OTC) on his or her own and without the obligatory assistance of authorized pharmacy personnel. While the herein depicted non-antibiotic treatments are presently available individually to a consumer without a prescription, it is the novel organization and conveyance of otherwise ordinary OTC agents that can now be seen to provide caregivers with an acceptable alternative to prescribing an antibiotic.

When primary care doctors were asked if they thought that their patients would be amenable to accepting such a non-antibiotic sinusitis package that was pharmacy-dispensed as an alternative to receiving an antibiotic prescription, their overall expectation was for approximately half of their patients to be amenable. Table 2 shows the distribution of this expectation among responding doctors, and it can be seen that 77% of the doctors thought that the proposed non-antibiotic treatment packages would be acceptable to 50% or more of their patients.

TABLE 2

| Percent of | % doctors |
|---|---|
| patients | reporting |
| 100% | 2% |
| 75% | 28% |
| 50% | 47% |
| 25% | 19% |

It therefore appears that the simple provision for availability of such a package would substantially reduce antibiotic prescribing for acute sinusitis.

When further asked about patient acceptance if cost for the package were reimbursed by insurance as is ordinary for prescription drugs, the doctors overall expectation was for two-thirds to three-quarters of patients to find it acceptable (two surveys). Table 3 shows the distribution of this expectation among doctors in one survey, and it can be seen that 75% of doctors thought that the proposed non-antibiotic treatment package would be acceptable to 75% to 100% of their patients if it's cost were reimbursed by insurance.

TABLE 3

| Percent of | % doctors |
|---|---|
| patients | reporting |
| 100% | 15% |
| 75% | 60% |
| 50% | 11% |
| 25% | 10% |

Incentives to encourage patient acceptance are herein desired and insurance reimbursement is therefore preferred.

It is one intention that packages of the present invention be devised according to cost and reimbursed such that the patient pays less for non-antibiotic treatment than for antibiotic treatment, and considered that insurers may find incentive to reimburse patients for the cost of non-antibiotic packages for cost savings, but also to encourage antibiotic conservation. Presently, the treatment of resistant organisms costs the United States healthcare system over $20 billion dollars a year, and there is incentive to avoid yet larger future costs,

The present invention further contemplates a method that encourages non-antibiotic treatment when the clinical presentation is likely viral, but also provides an antibiotic for the contingency of bacterial infection developing after the initial clinical encounter. The provision of both a non-antibiotic treatment package and an antibiotic treatment package allows such a method.

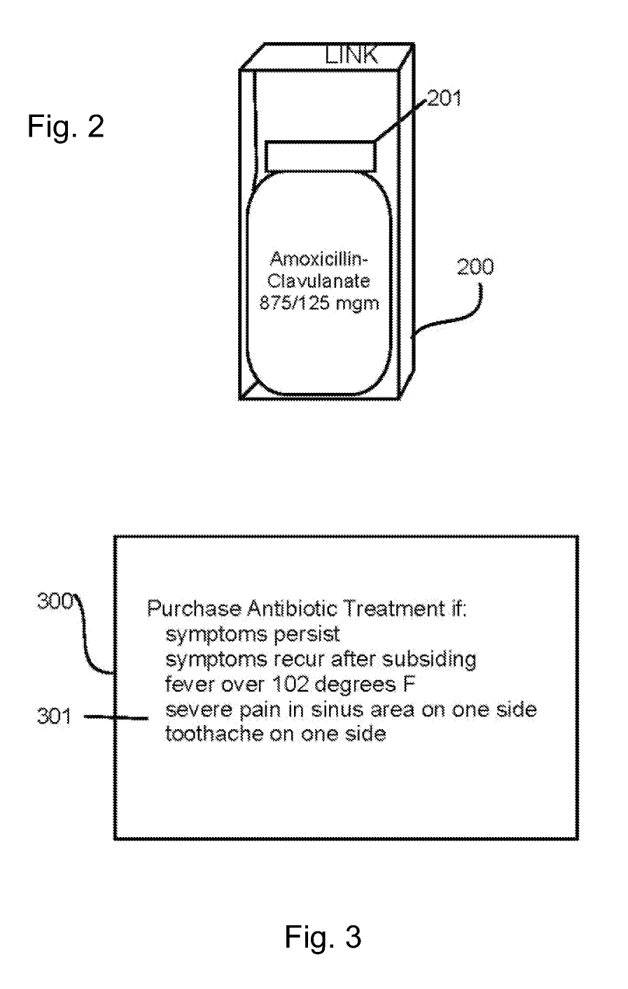

FIG. 2 schematically depicts an embodiment of an antibiotic package or module 200 of the present invention that is filled by prescription, and utilized if needed in the event of bacterial sinusitis. It is a further intention of the present invention to provide an appropriate antibiotic for acute bacterial sinusitis for when an antibiotic is indicated, that is, an antibiotic that is as effective and specific for the intended organism(s) as possible, rather than one having a broad range of antimicrobial activity, where broader emergence of resistant organisms might unnecessarily occur. In this case the illustrated antibiotic medication 201 is amoxicillin-clavulanate. It is further contemplated for symptomatic medication to be included in such antibiotic packages as might be considered desirable.

Means for packaging medication is well known in the art and conventional manufacturing, packaging, and distribution by a pharmaceutical manufacturer, packager and/or distributor (and not a physician, pharmacy, or patient) is acceptable. It can be observed that trade dress of prescription drugs is generally plainer than that of over-the-counter drugs, where OTC packages are intended to stand out to attract sales from among a many displayed items. It is further option that the trade dress of the packages of the present invention, both non-antibiotic and antibiotic, be devised to have an appearance commensurate with prescription packaging rather than over-the-counter packaging that is devised to capture attention on an OTC shelf.

In another embodiment of the present invention, non-antibiotic and antibiotic packages appear visually related to each other by any of logo, wording, color, design, and/or indicia (herein illustrated by the word “LINK”) that are exclusive and distinct from other pharmaceutical products.

FIG. 3 illustrates another embodiment of the present invention that contains an optional constraint directive 300 that is essentially a written checklist or algorithm to stipulate the circumstances 301 for when antibiotic acquisition and use is advised. The constraint directive 300 might constitute an advisory sheet that a physician or pharmacist can convey to the patient. Most particularly, the constraint directive 300 is incorporated within a non-antibiotic package, preferably on a prominent surface (illustrated as 103), such that the patient is readily informed of the clinical contingencies that warrant reaching out to a caregiver and/or filling a prescription for an antibiotic. The illustrated contingencies to fill the antibiotic are, in this case for: prolonged symptoms, worsening of symptoms after a period of improvement, fever of over 102 degrees, severe unilateral pain in the region of the nasal sinuses, and unilateral toothache.

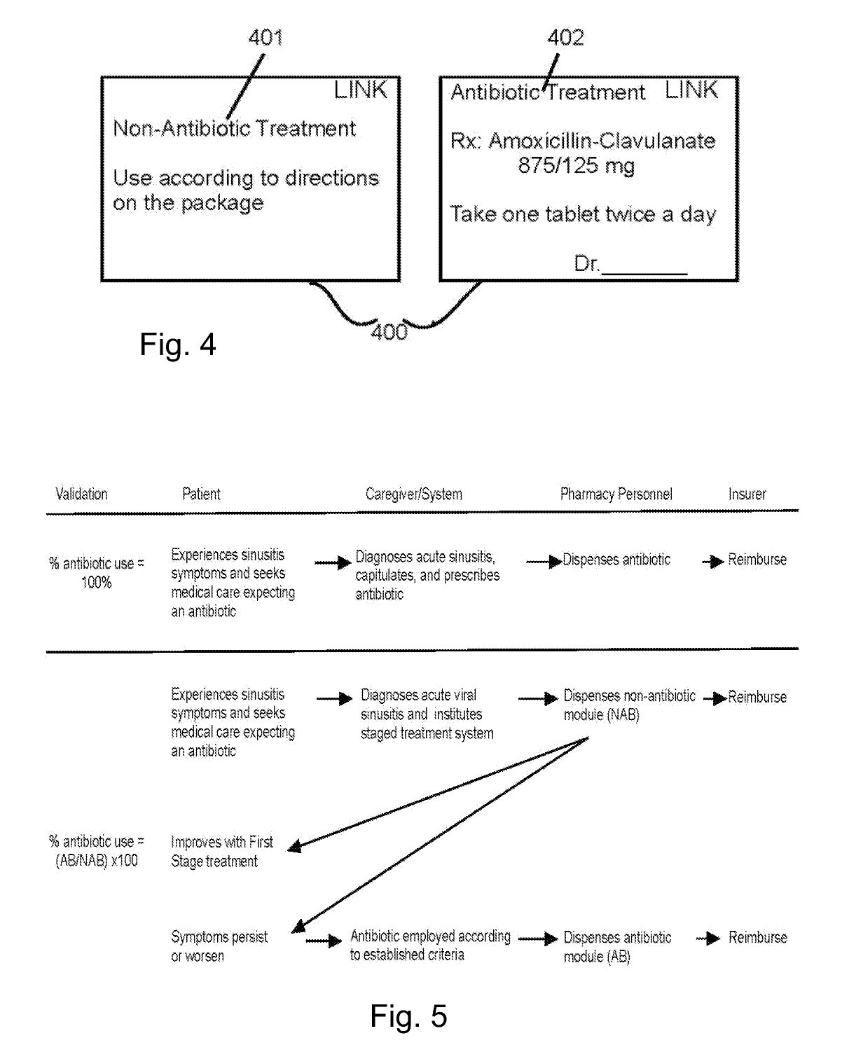

FIG. 4 illustrates a further aspect of the present invention that is an optional procurement means 400 for a provider to initiate patients' procurement of treatment packages of the present invention. The intent of the present invention to establish the acquisition of the non-prescription treatments as similar to acquiring a prescription drug, and it is therefore preferred for the procurement means to be in written form as is a prescription, rather than for the caregiver to verbally recommend the non-antibiotic package. It is also another advantage of the present invention to provide for efficiency for a caregiver to explain treatment to a patient. The illustrated non-prescription procurement means 401 for procuring the non-antibiotic package includes the name the package and instructions for its use, or it might alternately refer the patient to directions on the package or within its packaging (102 herein depicted on the container of a therapeutic entity). In other embodiments, non-prescription procurement means 401 of the present invention further incorporate a constraint directive 300 that informs the user of contingencies for which to either seek further help, or use antibiotic (not illustrated).

The second illustrated procurement means is a conventional prescription 402 for an antibiotic package that is only dispensed at a pharmacy by prescription. The prescription 402 is illustrated as requiring a caregiver's signature and further as providing instructions for use, or it might alternately direct the patient to use the antibiotic according to conventional package directions (not illustrated in FIG. 2).

While the procurement means 400 is depicted as two separate documents 401 and 402, it is alternatively considered that the procurement means 400 may constitute a single sheet or single dividable sheet (as scored) that advises obtaining the non-antibiotic module and also incorporates the antibiotic prescription for use later if needed.

In another embodiment of the present invention, procurement means incorporates indicia as wording, logo, color, design, and/or indicia (herein illustrated by the word “LINK”) that visually link both the procurement means and the non-antibiotic and antibiotic modular packages as a coordinated system for when they are employed together. Such visual cues are also incorporated on the packaging of the modules (illustrated by the word “LINK”) and/or medications and are not otherwise employed for other products by a manufacturer or distributor of the packages. The procurement means may be devised, and made available to prescribers by manufacturers and/or distributors of the treatment packages, but it is also contemplated that caregivers might themselves devise procurement means to convey to patients.

It is a further intention of the present invention to encourage non-antibiotic treatment for acute sinusitis even for those patients that most strongly insist upon receiving an antibiotic, as varying degrees of determination to receive antibiotic among patients is contemplated. It is therefore a method of the present invention for a caregiver to choose to convey both non-prescription and prescription procurement means at an initial clinical encounter for especially insistent patients, such that the patient first acquires the non-antibiotic treatment package, but is assured of the availability of antibiotic later should symptoms develop that more clearly indicate antibiotic need than at the time of seeing the caregiver. When caregivers were asked to describe the patient acceptability of a pharmacy-dispensed non-antibiotic package if the patient were also provided with an antibiotic prescription to use contingent to worsening, their overall expectation was that about two-thirds to three-quarters of their patients would find this acceptable (two surveys). Table 4 shows the distribution in one study of the doctors' expectation for acceptance of the non-antibiotic packages in the event of the patient also being provided with a contingent antibiotic prescription. It can be seen that 73% of doctors thought that three quarters or all of their patients would find this acceptable.

TABLE 4

| Percent of | % doctors |
|---|---|
| patients | reporting |
| 100% | 35% |
| 75% | 38% |
| 50% | 19% |
| 25% | 6% |

While the intent of the present invention is to minimize clinical interactions such as repeat calls and visits that can burden the doctor and healthcare system by anticipating the need for contingent treatment, it is however acknowledged that some doctors may prefer to actively validate antibiotic need and provide an antibiotic prescription only after they personally establish that the conditions for antibiotic employment have been met.

FIG. 5 is a flow-sheet illustration comparing current antibiotic use vs. antibiotic use according to the present invention. Antibiotic is employed 100% of the time where a caregiver is coerced by a patient expecting to receive an antibiotic, and lacking a practicable alternative, prescribes an antibiotic. Antibiotic is not employed at all where a caregiver initiates the staged treatment system, the pharmacy personnel conveys a non-antibiotic treatment package (NAB) to the patent and the patient improves. When symptoms persist or worsen, antibiotic is resorted to according to established criteria and dispensed by the pharmacy (AB). The ratio of AB/NAB can be used to validate restraint in antibiotic employment.

The staged treatment system method of the present invention can be seen to afford validation and quantification of reduction of antibiotic employment. Each instance where non-antibiotic treatment is used alone is an indication that antibiotic was not employed where it otherwise might have been. Means to track pharmaceutical sales are well known in the art, and can be employed to measure the relative employment of the non-antibiotic and antibiotic packages of the present invention to quantify the effectiveness of the system to restrain antibiotic use.

Validation of the effectiveness of the present method can be performed by sampling virtually any size cohort and does not necessarily require a large scale. Ready assessment of local, regional, or situational outcomes is an advantage of the present method, where such direct validation has not been possible with past strategies as educational initiatives, advisories, and published guidelines.

It is further contemplated that refinements to the system can be made if desired according to measured efficacy. Modifications in the therapeutic agents themselves, their dosing, packaging, adjustments in methods of prescribing and advising treatments (for example, revision of the formatting, appearance, or provisions of procurement means and/or constraint directives), and changes in dispensing to attempt to best encourage restraint and appropriate antibiotic use are contemplated.

Reimbursement for a non-antibiotic module is also illustrated in FIG. 5. Insurer reimbursement to encourage use of non-antibiotic medication is another element of the system, and incentives in the form of partial or total reimbursement for non-antibiotic therapeutics and/or providing for comparatively less reimbursement for antibiotic than non-antibiotic treatments are contemplated. Further contemplated is a system of “pending reimbursement” where antibiotic coverage is withheld pending the patient first trying non-antibiotic therapy, perhaps for even as short a period as a day.

The herein described treatments and embodiments are based upon the expectation among practicing doctors for significant patient acceptance and therefore utility to significantly reduce antibiotic utilization for acute sinusitis. The term “patient” is herein intended to refer to individuals seeking treatment, and the terms “caregiver,” “practitioner,” and “doctor” to include those who care for patients and are authorized to write drug prescriptions including physicians, physician assistants, and others acting for such prescribers. The term over-the-counter (OTC) is intended to refer to treatments that are dispensed without prescription at pharmacies where a lay individual can select and purchase them from the over-the-counter shelf without the intervention of pharmaceutical personnel as are required for dispensing prescription, or behind-the-counter pharmaceuticals. The term “pharmacy-dispensed” is herein intended to indicate conveyance of a treatment package located “behind-the-counter” to a patient or purchaser by a pharmacist, or by authorized pharmacy personnel that requires patient/purchaser identification and patient/purchaser signature in distinction to ordinarily procuring treatment OTC. The terms “package” and “module” are herein used interchangeably, as are the terms “sinusitis” and “rhinosinusitis” and the terms “antibiotic” and “antimicrobial agent” are herein intended to refer to an antibacterial drug and not an anti-viral agent.

The herein described treatments and embodiments that apply to acute sinusitis are not intended to limit the scope of the invention. Over 100 million antibiotic prescriptions are written in the US yearly, 41 million of them for respiratory conditions where their employment is often unnecessary. Similarly devised treatments are contemplated for other disease entities such as other respiratory tract infections where patient coercion for an antibiotic and need for an acceptable alternative to prescribing an antibiotic can be substantiated to underlie antibiotic overprescribing.

Although the preferred embodiments of the present invention have been described herein, the above description is merely illustrative. Further modification of the invention herein disclosed will occur to those skilled in the respective arts and all such modifications are deemed to be within the scope of the invention as defined by the appended claims.

What is claimed is:

1. A method to treat acute sinusitis as an alternative to prescribing an antibiotic or recommending over-the-counter medications, the method comprising:
providing a non-antibiotic treatment package containing a pharmacy-dispensed decongestant and an over-the-counter therapeutic agent for acute sinusitis that includes a topical nasal moisturizing means and at least one of an antihistamine, an oral mucolytic agent, an analgesic, a topical corticosteroid, a topical anticholinergic agent, an oral anticholinergic agent, a cough suppressant, an antiviral agent, a vitamin, an anti-inflammatory agent, a symptomatic vapor treatment and not an antibiotic, and further selecting pseudoephedrine that is pharmacy-dispensed wherein the non-antibiotic treatment package is a behind-the-counter treatment package;
advising a patient to procure the treatment package to treat acute sinusitis as an alternative to prescribing an antibiotic or recommending over-the-counter procurement of treatment; and
pharmacy-dispensing the treatment package to a patient.

2. The method of claim 1 further comprising recommending the treatment package to a doctor as an alternative to prescribing an antibiotic for acute sinusitis where the doctor does not have to direct patients to ordinarily procure over-the counter medications.

3. The method of claim 1 wherein the providing step includes devising a treatment package appearance commensurate with prescription packaging, and applying it to said pharmacy-dispensed non-antibiotic treatment package.

4. The method of claim 1 further comprising devising a non-prescription procurement means and conveying the non-prescription procurement means to a patient by a caregiver.

5. The method of claim 1 further comprising the steps of devising a restraint directive that directs a patient to employ an antibiotic only upon encountering at least one of prolonged symptoms, worsening of symptoms after a period of improvement, a high fever, severe unilateral pain in the region of the nasal sinuses, and unilateral toothache.

6. The method of claim 5 wherein the devising step includes incorporating the restraint directive upon a surface of at least one of the non-prescription procurement means and the non-antibiotic treatment package.

7. The method of claim 5 further comprising conveying the restraint directive to a patient by a caregiver.

8. The method of claim 1 further comprising reimbursing the patient the cost of the treatment package to persuade a patient to use the non-antibiotic treatment package.

9. The method of claim 1 further comprising providing an antibiotic treatment package for sinusitis wherein the antibiotic treatment package contains an antibiotic agent selected according to current antimicrobial sensitivities.

10. The method of claim 9, wherein the providing an antibiotic treatment package includes selecting antibiotic agent that is one of amoxicillin or amoxicillin-clavulanate.

11. The method of claim 9 further comprising linking the antibiotic treatment package to the non-antibiotic treatment package by at least one of a logo, wording, color, design, and indicia.

12. The method of claim 9 further comprising the steps of devising a prescription procurement means and conveying the prescription procurement means to a patient.

13. The method of claim 12 wherein the devising of the non-antibiotic procurement means and the antibiotic procurement means includes devising one of an individual and separate procurement means for each of the non-antibiotic treatment package and the antibiotic treatment package, a combined non-antibiotic and antibiotic procurement means, and a combined procurement means that are divisible.

14. The method of claim 13 further comprising conveying the antibiotic procurement means to a patient at the time of conveying the non-antibiotic procurement means to a patient to thereby encourage non-antibiotic treatment by providing for a contingent antibiotic need.

15. The method of claim 9 further comprising establishing insurance reimbursement for the antibiotic treatment package that is contingent upon a period of use of the non-antibiotic treatment package of at least one day to encourage non-antibiotic treatment of acute sinusitis.

16. The method of claim 9 further comprising quantifying restraint in antibiotic use by ascertaining the employment of the non-antibiotic treatment package relative to the antibiotic treatment package.

17. A method to treat acute sinusitis as an alternative to prescribing an antibiotic or recommending over-the-counter medications, the method comprising:
providing a non-antibiotic treatment package containing a pharmacy-dispensed decongestant and an over-the-counter therapeutic agent for acute sinusitis that includes a topical nasal moisturizing means, and further selecting pseudoephedrine that is pharmacy-dispensed wherein the non-antibiotic treatment package is a behind-the-counter treatment package;
advising a patient to procure the treatment package to treat acute sinusitis as an alternative to prescribing an antibiotic or recommending over-the-counter procurement of treatment; and
pharmacy-dispensing the treatment package to a patient.

18. The method of claim 17 wherein the providing step includes providing at least one of an antihistamine, an oral mucolytic agent, an analgesic, a topical corticosteroid, a topical anticholinergic agent, an oral anticholinergic agent, a cough suppressant, an antiviral agent, a vitamin, an anti-inflammatory agent, a symptomatic vapor treatment and not an antibiotic in the non-antibiotic treatment package.

19. The method of claim 17 further comprising devising a non-prescription procurement means and conveying the non-prescription procurement means to a patient by a caregiver.

20. The method of claim 19 further comprising recommending the treatment package to a doctor as an alternative to prescribing an antibiotic for acute sinusitis where the doctor does not have to direct patients to procure over-the counter medications and providing the doctor with the non-prescriptive procurement means indicating the treatment package for conveying to a patient.
